(19) United States
(12) Patent Application Publication
(10) Pub. No.: US 2023/0330003 A1
(43) Pub. Date: Oct. 19, 2023

(54) Compositions Comprising Silybum Marianum Extract As A Senotherapeutic Agent

(71) Applicant: Clariant International Ltd., Muttenz (CH)

(72) Inventors: Mathilde FRECHET, Toulouse (FR); Hanane CHAJRA, Tournefeuille (FR)

(73) Assignee: Clariant International Ltd., Muttenz (CH)

(21) Appl. No.: 18/030,176

(22) PCT Filed: Sep. 8, 2021
(86) PCT No.: PCT/EP2021/074651

§ 371 Date: Apr. 4, 2023

(30) Foreign Application Priority Data
Oct. 5, 2020 (EP) 20200056.8

(51) Int. Cl. A61K 8/9789; A61Q 19/08

(52) U.S. Cl. CPC A61K 8/9789; A61Q 19/08

(57) ABSTRACT

The present invention relates to the use of a composition comprising or consisting of an extract of flowers of Silybum marianum from which seeds have been removed comprising less than 0.1% by weight of silymarin based on the total weight of the dry extract, as a senotherapeutic agent for reducing cellular senescence in a tissue. Furthermore, the present invention refers to a composition comprising or consisting of an extract of flowers of Silybum marianum from which seeds have been removed as a senotherapeutic agent for use in a method for treating or preventing a disorder associated with cellular senescence in a tissue.

### FIELD OF THE INVENTION

The present invention relates to the (cosmetic) use of a composition comprising or consisting of an extract of flowers of Silybum marianum from which seeds have been removed, comprising less than 0,1% by weight of silymarin, based on the total weight of the dry extract, as a senotherapeutic agent for reducing cellular senescence in a tissue present in the skin. Furthermore, the present invention refers to a composition comprising or consisting of an extract of flowers of Silybum marianum from which seeds have been removed as a senotherapeutic agent for use in a method for treating or preventing a disorder associated with cellular senescence in a tissue present in the skin. Accordingly, the present invention refers to an active ingredient consisting of an extract of flowers of Silybum marianum from which seeds have been removed, comprising less than 0,1% by weight of silymarin, based on the total weight of the dry extract and its use. Particularly, the invention relates to an extract of flowers of Silybum marianum from which seeds have been removed, comprising less than 0.1% by weight of silymarin, based on the total weight of the dry extract for use as a senotherapeutic active ingredient in preventing and/or in treating age-related or stress-related senescent cells within a tissue; and/or in treating cellular senescence-associated disorders within a tissue; more generally in delaying and/or in preventing the apparition of signs of tissue aging and tissue deterioration.

### BACKGROUND OF THE INVENTION

Cellular senescence is a physiological process and a tumour-suppressive cell fate characterized by a permanent and irreversible cell cycle arrest as well as acquisition of a pro-inflammatory and proteolytic secretome. Chronological aging is typically correlated with an increase of senescent cells, including skin cells. Indeed, skin is the human body's first barrier. It protects the organs from temperature and humidity differences and from attack by the external environment. However, excessive chemical and physical stimulations deteriorate the normal functions of the skin and induce aging thereof.

Skin aging is a multi-factorial process drives by both intrinsic (e.g.: time, genetic factors, hormones) and extrinsic (e.g., UV-exposure, pollution) factors. Characteristics of intrinsic or chronological aging include visible signs such as thin and dry skin, fine wrinkles, decreased elasticity, aberrant pigmentation, hair greying, and hair loss.

Among which epidermal thinning causes, decreased proliferation and renewal capacity of basal keratinocytes has been reported. Besides the epidermis, both the dermal epithelial junction (DEJ) and the dermis often also become thinner. Fibroblasts residing within the dermis generate extracellular matrix (ECM) that provides the skin with structural integrity and elasticity. During aging, the ECM typically undergoes structural alterations and degradation, leading to the increase of wrinkles and loss of elasticity of the skin. In adult tissues, senescence is activated upon cellular damage as a defence mechanism, not only against oncogenic unlimited proliferation signal that drives cancer but contributes also to the physiological processes of normal organismal aging in many cell types in response to diverse stress. Cellular senescence often promotes tissue remodelling through three sequential processes: a stable proliferative arrest, a SASP that recruits immune cells and modifies the extracellular matrix, and the mobilization of nearby progenitors that repopulate the tissue.

Markers of senescence are described in the age-related skin alteration process. Among them, the plasminogen activator inhibitor type 1 (PAI-1), an inhibitor of serine proteases, is elevated in dermal fibroblasts derived from patients with premature aging syndrome and chronologically aged donors. Moreover, senescence marker studies reported that PAI-1 is a mediator of cell cycle arrest by its role in triggering senescence downstream of the essential cell cycle checkpoint p53.

Senescent cells present characteristic morphological features, high activity of senescence-associated beta-galactosidase (SA-β-gal), increase of senescence-associated secretory phenotype (SASP) such as cytokines and matrix metalloproteinase, reduction of lam in B1 expression and translocation of nuclear HMGB1 into the cytoplasm (Wang and Dreesen, 2018: Frontiers in Genetics, Vol. 9, Article 247). Moreover, the permanent growth arrest is driven by telomere shortening and a cell cycle blockade initiated by an enhanced p53 activity, increased p21 and p16INK4A-Rb expression (Lozano-Torres et al., 2019: Nature Reviews in Chemistry, Vol. 3, pages 426-441).

In addition to the loss of proliferative ability, senescent cells often bear enhanced survival and resistance to apoptosis through the activation of various mechanisms referred to as senescent cell anti-apoptotic pathways (SCAPs). These pathways are directly related to many features among which growth arrest, metabolic reprogramming, intrinsic, and extrinsic apoptotic pathways.

Senescent cells exert pleiotropic effect that could be explained by their ability to secrete an array of inflammatory cytokines (e.g: IL-1α, IL-1β, IL-6, IL-8, (gamma-HMGB1), chemokines (e.g., CXCR2), growth factors (e.g: IGFBP7), and proteases such as the metalloproteinases (e.g., matrix metalloproteinases (MMP-1 and MMP-3) known as SASP. Depending on the physiological context, the SASP secretion can be either beneficial or deleterious especially when persistent senescent cells negatively impact the surrounding microenvironment by impairing tissue homeostasis. Indeed, some SASP components such as IL-6 and IL-8 can reinforce SAGA (senescence associated growth arrest) through autocrine pathway mediated by secreted and membrane-bound IL-1α. However, the same secreted components can act in paracrine signalling to neighbouring cells, propagating the senescent phenotype and thus potentially hampering the regenerative capacity of surrounding tissue. Moreover, SASP cytokines promote infiltration of immune cells, leading to a persistent and chronic inflammation, a hallmark of aging and major contributor to age-related dysfunctions. In vivo, elevated IL-6 has been detected in nevi melanocytes while MMPs are detected in chronologically aged and photo-aged skin and are responsible for breakdown of the extracellular matrix (ECM). In senescent cells, HMGB1 translocates from the nucleus to the cytoplasm and extracellular space, facilitating the release of SASP factors including IL-113, IL-6 and MMP-3. The SASP secretion is associated with senescence and could be detached from the cell cycle arrest. Indeed, studies in primary human fibroblasts demonstrated that DNA damage leads to a SASP independent of p16INK4A levels. One critical actor for the transcription of SASP is the phosphorylation of the transcription factor NF-kB which translocate to the nucleus where it binds the promoters of several SASP genes.

Senolytics, which are typically understood as agents able to selectively kill senescent cells, were the first potential senotherapy to be successfully tested in preclinical in vivo models. In other words, an agent is considered as having senolytic activity when its activity includes specifically inducing or promoting cell death, in particular apoptotic cell death, of senescent cells in the tissue. Several senolytic agents have now been identified including quercetin, which inhibits the PI3K pathway and acts on Bcl-2 family, p53/p21, dasatinib which interferes with dependence receptor EFNB; ABT-763; and navitoclax, also called ABT-263, which targets the Bcl-2/Bcl-xL proteins. Others senolytic agents are peptide FOXO4-D-Retro Inverso and 17-DMAG (alvespimycin). Such senolytic agents are able to selectively kill senescent cells by induction of apoptosis process, but not on non-senescent cells or normal cells. To date, studies on agents that selectively kill senescent cells have been focused only on single compounds, but little research has been carried out on a mixture of compounds such as plant extracts for example.

Senomorphics are typically understood as agents that suppress markers of senescence or their secretory phenotype without inducing apoptosis, providing a senomorphic effect by a mechanism different to that of senolytics. In other words, an agent is considered as having senomorphic activity when its activity includes suppressing senescence phenotypes in senescent cells in the tissue.

According to the literature, the above senolytic agents were not described as enabling to exert senomorphic activity on senescent cells.

According to the above-referenced, preventing occurrence or accumulation of senescent cells within tissues appears is a strategy of interest to prevent or treat age-related or stress-related tissue deterioration caused by senescence cells. Indeed, senescent fibroblasts represent between 20 and 60% of the total skin cells. However, the blockage of p16INK4A and p53 or activating telomerase to extend the proliferative capacity of cells inevitably leads to an increased cancer risk. Additionally, MMP-1 and MMP-3 or proinflammatory cytokines such as IL-6 and IL-8 secreted from senescent fibroblasts are known as SASP playing a critical role in degradation of ECM leading to worsening skin conditions. Thus, a balanced approach ensuring the prevention of increase number of senescent cells and simultaneously limiting the extension of proliferative capacity of cells should be considered to fight age-related and stress related cellular senescence within a tissue.

Additionally, selectively eliminating senescent cells can increase lifespan and can improve health span and may be beneficial for a wide range of disease treatments and preventions (Gonzalez-Gualda et al., 2020, Aging Cell. 2020; 19:e13142). These findings provide plausibility that suppression of senescent cells is a good target to counteract aging process and tissue deterioration. As a consequence, a credible approach is consisting of selectively eliminating senescent cells and/or modulating their function, particularly the SASP.

Some senotherapeutic agents, including senolytic and senomorphic agents are known in the art.

US-A 2019/054097 disclosed a number of rather complex synthetical small-molecular senolytic agents, similar to ABT-263, selectively killing senescent cells. These compounds enable interference with the anti-apoptotic Bcl-2 pathway that is also involved in cancer. WO 2020/122392 disclosed the rather complex immunosuppressant zotarolimus, which is a derivative of sirolimus (rapamycin), for use as a senomorphic agent recovering senescent cells. WO 2020/132053 disclosed dasatinib and quercetin as senotherapeutic agents for treating obesity-induced neuropsychiatric disorder like anxiety. These therapeutic agents may bear severe undesired side effects.

US-A 2020/121620 teaches senolytic agents for killing senescent cells with a focus on synthetical small-molecular drugs typically used for therapeutic applications in other fields. Also KR-A 2020/0072924 teaches senolytic agents for killing senescent cells, including fibroblasts.

A drawback of these aforementioned agents and compositions comprising such agents is that these are focused on specific agents. These are often of synthetic origin, have rather complex chemical structures which require significant efforts to be obtained, and have severe undesired side effects.

Therefore, it is desirable to obtain further compositions having a senotherapeutic which are of natural origin, having reduced undesired side effects and are obtainable at low efforts.

Nature provides a wide variety of plant-based agents and compositions, including plant extracts.

The plant Silybum marianum is traditionally known to be effective for improving liver function, for reducing total cholesterol amount, for treating diabetes, for preventing motion sickness and heart diseases. Silybum marianum is sometimes also designated as (blessed) milk thistle. On the other hand, the name “milk thistle” is ambiguous. It may refer to different species such as Lactuca serriola, various Sonchus species (e.g., Sonchus asper, Sonchus arvensis, Sonchus oleraceus), and Silybum marianum.

It was also considered for changing the appearance of the skin. EP-B 1 845 935 teaches that the compound silymarin or isosilybin can have numerous effects, including influencing pigmentation of hair and skin. EP-B 2 658 522 teaches topical administration of combinations of Silybum marianum and Momordica grosvenori extracts having antioxidant effects and reducing cellular stress factors. US-A 2010/316743 teaches that a milk thistle extract, beside numerous other agents, can be used for promoting the inhibition of the formation of advanced glycation end products (AGEs). CN-A 103961276 teaches milk thistle extract, beside numerous other plant extracts, for an anti-glycation cosmetic composition. CN-B 104382833 teaches a plant peptide solution, which may contain, inter alia, milk thistle extract as an anti-wrinkle and skin-improving agent. JP-A 2002/034505 teaches a food composition having reduced bitterness and contains a polysaccharide and silymarin which can be extracted from a milk thistle. US-A 2019/175677 teaches a Silybum marianum achene extract, comprising less than 0.2% of silymarin, for use in the treatment of acne, seborrhea, seborrheic dermatitis and/or rosacea. WO 2014/151891 disclosed Nrf2-activating agents containing plant extracts including a milk thistle extract. This document also mentions Silybum marianum seed extract which may contain 80% by weight of silymarin as active ingredient. EP-B 0 180 505 teaches silymarin which can be extracted from a milk thistle for obtaining anti-inflammatory effects. Senotherapeutic uses, in particular evolving senolytic and/or senomorphic activities, i.e., targeting senescent cells within a tissue, do not seem to have been considered. KR-A 20200063855 teaches a hydroethanolic extract of the flowers devoid of seeds of Silybum marianum for promoting the melanin production, for preventing vitiligo, for hair greying and hypopigmentation. However, the senotherapeutic effects as described above are not disclosed. The hydroethanolic extract of the flowers of Silybum marianum, containing silibinin (main component of silymarin) is described to inhibit the formation of glycated proteins (protection of collagen and elastin) within the skin by scavenging the free radicals (Seoungwoo Shin et al., Molecules 2015, 20, 3549-3564; doi:10.3390/molecules20033549).

There is still an unmet need for senotherapeutic compositions which are of natural origin, have low undesired side effects, and which are obtainable at low efforts.

### SUMMARY OF THE INVENTION

Surprisingly, it has been found that an extract of flowers devoid of seeds of Silybum marianum can be used as a senotherapeutic agent. Further, it has been surprisingly found that compositions comprising (or even consisting of) at least one extract of flowers devoid of seeds of Silybum marianum as a senotherapeutic agent can be used for reducing cellular senescence in a tissue and for treating or preventing a disorder associated with cellular senescence in a tissue present in the skin.

Accordingly, the present invention relates to the (optionally cosmetic) use of an extract flowers of Silybum marianum from which seeds have been removed as a senotherapeutic agent.

The present invention also refers to a cosmetic skin and scalp care method in preventing and/or in treating age-related or stress-related senescent cells in the skin and hair follicle; and/or in treating cellular senescence-associated disorders in the skin and hair follicles by applying, onto the skin or the scalp in need, at least an effective amount of an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract; or a composition comprising at least said extract.

A first aspect of the present invention relates to the (optionally cosmetic) use of a composition comprising (or consisting of) an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, as a senotherapeutic agent for reducing cellular senescence in a tissue present in the skin.

Accordingly, the present invention also relates to a method (which is optionally a cosmetic method) for reducing cellular senescence in a tissue, said method comprising administering a sufficient amount of a composition comprising (or consisting of) an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, as a senotherapeutic agent to said tissue.

Accordingly, the present invention also relates to a method (which is optionally a cosmetic method) for reducing cellular senescence in a tissue present in the skin of a subject, said method comprising administering a composition comprising (or consisting of) an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, as a senotherapeutic agent to said tissue of said subject.

A further aspect of the present invention relates to a composition comprising or consisting of an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, as a senotherapeutic agent for use in a method for treating or preventing a disorder associated with cellular senescence in a tissue present in the skin.

Accordingly, the present invention also refers to a composition comprising an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, for use as a senotherapeutic active ingredient in preventing and/or in treating age-related or stress-related senescent cells in a tissue present in the skin; and/or in treating cellular senescence-associated disorders in said a tissue.

Accordingly, the present invention also relates to a method for treating or preventing a disorder associated with cellular senescence in a tissue present in the skin, said method comprising administering a sufficient amount of a composition comprising (or consisting of) an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, as a senotherapeutic agent to said tissue.

Accordingly, the present invention also relates to a non-therapeutic method for treating or preventing a disorder associated with cellular senescence in a tissue present in the skin in a subject, said method comprising administering a sufficient amount of a composition comprising (or consisting of) an extract of flowers of Silybum marianum from which seeds have been removed as a senotherapeutic agent to said tissue in said subject.

Accordingly, the present invention also relates to a method for treating or preventing a disorder associated with cellular senescence in a subject, said method comprising administering a sufficient amount of a composition comprising (or consisting of) an extract of flowers of Silybum marianum from which seeds have been removed as a senotherapeutic agent to said subject.

The present invention also refers to the cosmetic use of an an extract of flowers of Silybum marianum from which seeds have been removed, or a composition comprising at least said extract, as a senotherapeutic active ingredient intended to prevent and/or to treat age-related or stress-related senescent cells within a tissue, preferably in the skin and hair follicle; and/or in treating cellular senescence-associated disorders within a tissue, preferably in the skin and hair follicles.

It was found that an extract of flowers devoid of seeds of Silybum marianum of the present invention exerts new beneficial effects on different tissues compared to those described in the prior art. Indeed, it has been demonstrated that an extract of Silybum marianum from which seeds have been removed, is enabling, firstly to selectively kill senescent cells (e.g., skin and hair follicle cells). Thus, the said extract of Silybum marianum was found to have senolytic activity. Secondly such extract was found to modulate phenotype of senescent cells, and help those cells to get rid of these deleterious cells. Thus, the said a extract of Silybum marianum was found to have senomorphic activity. This may include modulation of SASP secretion. Also the proliferation ability of those senescent cells may be involved.

### BRIEF DESCRIPTION OF FIGURES

FIG. 1 (A, B, C) demonstrates the senolytic activity of the Silybum marianum flower extract (SME) in senescence replicative model of human dermal fibroblasts (HDF).

FIG. 1A shows cell viability of non-senescent and senescent cells which were untreated or treated with ABT-737 or SME was compared. FIG. 1B shows the number of SA-β-Gal positive cells, wherein senescent cells treated with ABT-737 were compared to those treated with different concentrations of SME. FIG. 1C shows the reduction of p21 expression in senescent cells treated with ABT-737 were compared to those treated with different concentrations of SME.

FIG. 2 (A, B, C) relates to the senolytic activity of Silybum marianum flower extract (SME) in senescence replicative model of HDF by induction of apoptosis through caspase-3/PARP pathway specifically in senescent HDF. FIG. 2A shows apoptosis in non-senescent and senescent cells treated with ABT-737 or SME. FIG. 2B shows cleaved caspase-3 in non-senescent and senescent cells treated with ABT-737 or SME. FIG. 2C shows cleaved poly-ADP-ribose polymerase (PARP) in non-senescent and senescent cells treated with ABT-737 or SME.

FIG. 3 (A, B, C, D) relates to the senomorphic activity of Silybum marianum flower extract (SME) in senescence replicative model of HDF by inhibition of SASP factors release. FIG. 3A shows p-H2AX expression in senescent cells treated with ABT-737 or SME. FIG. 3B shows TNF-alpha expression in senescent cells treated with ABT-737 or SME. FIG. 3C shows IL-6 expression in senescent cells treated with ABT-737 or SME. FIG. 3D shows MMP-1 secretion in senescent cells treated with ABT-737 or SME.

FIG. 4 (A, B) relates to the senomorphic activity of Silybum marianum flower extract (SME) in senescence replicative model of HDF by the restoration of the proliferation ability of the senescent HDF. FIG. 4A shows percent cell proliferation in senescent cells treated with ABT-737 or SME. FIG. 4B shows doubling time of cells in senescent cells treated with ABT-737 or SME.

FIG. 5 relates to the senomorphic activity of Silybum marianum flower extract (SME) in senescence replicative model of HDF by the restoration of the collagen type I synthesis ability of the senescent HDF.

FIG. 6 relates to the senolytic activity of Silybum marianum flower extract (SME) in senescence replicative model of human follicles dermal papilla cells (HFDPC).

### DETAILED DESCRIPTION OF THE INVENTION

A first aspect of the present invention relates to the (optionally cosmetic) use of a composition comprising (or consisting of) an extract of flowers of Silybum marianum from which seeds have been removed, comprising less than 0.1% by weight of silymarin, or less than 0.01% by weight of silymarin, or less than 0.001% by weight of silymarin, or 0% by weight of silymarin, based on the total weight of the dry extract, as a senotherapeutic agent for reducing cellular senescence in a tissue present in the skin.

More generally, the extract according to the present invention may enable delaying and/or preventing the apparition of signs of tissue aging and tissue deterioration. It will be understood that, in a composition consisting of an extract of flowers of Silybum marianum from which seeds have been removed, the composition is the extract.

Notably, the experimental in vitro studies provided in this application demonstrated that the extract of flowers devoid of seeds of Silybum marianum according to the invention fulfils senolytic and/or senomorphic activities by targeting senescent cells, particularly skin and hair follicle cells. More advantageously, the said extract of Silybum marianum, exerts senolytic and senomorphic activities on the said senescent cells. As a consequence, the said extract of Silybum marianum according to the present invention can be used as agent enabling to comply with the senotherapeutic approach.

The plant Silybum marianum is belonging to the genus Asteraceae white patterned thistle is a perennial or biennial plant which leaves typically have clear white stripes. Originally, this plant is a native of southern Europe through to Asia. It is now found throughout the world. The generic name “Silybum” is from the Greek name by Dioscorides, and it is widely known as blessed milk thistle because of the milky sap comes out from a torn leaf. Silybum marianum is traditionally known to be effective for improving liver function, for reducing total cholesterol amount, for treating diabetes, for preventing motion sickness and heart diseases.

As used throughout the present invention, the extract of flowers devoid of seeds of Silybum marianum means flowers of Silybum marianum from which seeds have been removed. Preferably, the term “flower” as used herein refers to essentially only flower of Silybum marianum from which seeds have been removed.

The extract according to the present invention may be obtained by any means. It may optionally be obtained by a commercial supplier or may be partly or completely prepared from a Silybum marianum plant or one or more parts thereof.

The extract may be any kind of extract. It may be prepared from the fresh flowers of Silybum marianum, or may be prepared from the dried flowers of Silybum marianum, or may be prepared from the frozen or freeze-dried flowers of Silybum marianum. In both cases, seeds have been removed.

Preferably, the flowers of Silybum marianum may be used in chopped, blended or pulverized form. In a preferred embodiment, the extract is prepared from the dried flowers of Silybum marianum, in particular from dried and pulverized flowers of Silybum marianum.

As used herein, the term “extract” may be understood in the broadest sense as generally understood in the art. An extract may be any substance made by extracting a part of a raw material (here: flower of Silybum marianum from which seeds have been removed). Typically, extracting is achieved by using a solvent. Accordingly, in a preferred embodiment, the extract is obtained from solvent extraction methods including cold or hot extraction, ultrasonic extraction, reflux cooling, needle extraction and microwaves extraction. In a preferred embodiment, the extract is obtained by performing an ultrasonic extraction method.

Herein, a solvent may be added to the flowers of Silybum marianum and left together for a time as described herein. In a further step, the solid parts are preferably separated by the primary extract. This may be achieved by any means such as, e.g., filtration, sieving, ultrafiltration, cross-flow filtration, centrifugation, precipitation over time, or a combination of two or more thereof.

One or more solvents may be used for extraction. Such solvent may be any solvent suitable for this purpose. Typically, a solvent is liquid under the conditions it is used for extraction. A solvent may be selected from the group consisting of an organic solvent (e.g., an alcohol (e.g., methanol, ethanol, propanol, butanol, pentanol, phenol, glycerol, 1,3-butylene glycol, propane diol, etc.), water (including hot water or subcritical water), aqueous buffer, and a combination of two or more thereof. An organic solvent may be aliphatic or aromatic. Preferably, the solvent may be hydrated.

The flower extract of Silybum marianum according to the present invention may comprise any content of silymarin.

The component “silymarin” may be understood as generally understood in the art. It may be understood as a mixture of four flavonolignans which are silybin (also called silibinin), isosylibin, silychristin, and silydianin.

In a preferred embodiment of the present invention, the extract comprises less than 0.1% by weight of silymarin, or less than 0.01% by weight of silymarin, or less than 0.001% by weight of silymarin, or 0% by weight of silymarin, based on the total weight of the dry extract.

In a preferred embodiment, the flower extract of Silybum marianum is a hydroalcoholic or an alcoholic extract. Preferably, the extract is a hydroalcoholic extract.

It will be understood that also an hydroalcoholic or an alcoholic extract may also be a paste or solid from which the water and alcohol content have been partly or completely removed in one or more further steps subsequent to the extraction step. In a preferred embodiment, the hydroalcoholic or an alcoholic extract is a dry powder.

In a preferred embodiment, the flower extract of Silybum marianum is a hydroethanolic extract obtained by extraction with an ethanol/water mixture containing between 10 and 90% (v/v) ethanol, or containing between 25 and 85% (v/v) ethanol, or containing between 50 and 82% (v/v) ethanol, or containing between 60 and 80% (v/v) ethanol, or containing between 65 and 75% (v/v) ethanol, or containing approximately 70% (v/v) ethanol.

It will be understood that also an hydroethanolic or an ethanolic extract may also be a paste or solid from which the water and ethanol content have been partly or completely removed in one or more further steps subsequent to the extraction step. In a preferred embodiment, the hydroethanolic or an ethanolic extract is a dry powder.

Extraction may be performed at any temperature suitable for this purpose. Preferably, extraction is conducted at a temperature in the range of 10 to 150° C., or 15 to 100° C., or 20 to 80° C., or 25 to 70° C. or 15 to 25° C., or approximately 20° C. In a preferred embodiment, extraction is conducted at ambient temperature (i.e., 15 to 25° C., in particular approximately 20° C.).

In a preferred embodiment, extraction is conducted at ambient temperature (i.e., 15 to 25° C., in particular approximately 20° C.) and ambient pressure (i.e., 980 to hPa).

In a preferred embodiment, extraction is conducted at ambient temperature (i.e., 15 to 25° C., in particular approximately 20° C.) and ambient pressure (i.e., 980 to hPa) using an ethanol/water mixture containing between 65 and 75% (v/v) ethanol, in particular approximately 70% (v/v) ethanol.

In a preferred embodiment, the flower extract of Silybum marianum comprises an extract of flowers of Silybum marianum from which preferably seeds have been removed, and extraction is conducted at ambient temperature (i.e., 15 to 25° C., in particular approximately 20° C.) and ambient pressure (i.e., 980 to 1200 hPa) using an ethanol/water mixture containing between 65 and 75% (v/v) ethanol, in particular approximately 70% (v/v) ethanol.

The extraction may be conducted for any time interval suitable for this purpose. For example, the extraction may be conducted for a time interval in the range of less than 30 min, of 30 min to 1 hour, of 1 hour to 2 hours, of 90 min to 6 hours, of hours to 24 hours, of 6 hours to 36 hours, of 12 hours to two days, of one day to a week, of a week to a month, or of more than a month. In a preferred embodiment, extraction may be conducted (preferably at ambient temperature of 15 to 25° C.) for a time interval in the range of 6 hours to 18 hours, of 20 hours to two days, exemplarily approximately between 10 hours and 90 hours, between hours and 70 hours, or between 40 hours and 60 hours. It may be conducted over night.

Accordingly, the method for preparing an extract of Silybum marianum according to the present invention, comprises the steps of:
(i) providing the flowers devoid of seeds (optionally dried) of Silybum marianum,
(ii) contacting the said flowers with at least an alcoholic solvent, wherein:
   (a) the alcoholic extraction is being performed at 15 to 60° C., preferably at 20 to 50° C., even more preferably at 25 to 30° C., for 1 hour to 24 hours, preferably for 2 hours to 20 hours, most preferably for 5 to 15 hours; and/or;
   (b) a hydroalcoholic ultrasonic extraction is conducted at 15 to 100° C., preferably at 15 to 80° C., most preferably at 15 to 50° C. for 1 hour to hours, preferably for 2 hours to 20 hours, most preferably for 5 to hours;
(iii) separating the extract from the solid residuals of step (ii); and
(iv) optionally concentrating the extract by removing the at least one solvent or parts thereof.

The extract according to the present invention may be liquid, pasty or solid at standard conditions (room temperature of approximately 20° C., normal pressure of approximately 1013 hPa). In this context, the terms “liquid” and “fluid” may be understood interchangeably. Preferably, the extract is first obtained in liquid form. Then, the one or more solvents may optionally be removed to obtain a solid form of the extract. A liquid extract can also be concentrated. Then, preferably, parts of the one or more solvents are removed. Optionally, a liquid or pasty extract may be obtained. Optionally, the extract may also be diluted with one or more solvents. Optionally, the extract may also be purified by any means (e.g., crystallization, chromatographic means, etc.). An extract may be optionally designated as “tincture” or “absolute”.

The flower extract of Silybum marianum of the present invention may be optionally obtained in a fraction produced by conducting other purification methods including separation through an ultrafiltration membrane with a constant molecular weight cut-off value, or ionic exchange, or separation through various chromatographic methods, or liquid-liquid separation or crystallization or precipitation.

For example, the flower extract of Silybum marianum of the present invention may be prepared by drying the flowers devoid of seeds of Silybum marianum, pulverizing it and then extracting it with, an alcohol or a hydrated alcohol, followed by filtration under reduced pressure.

In a preferred embodiment of the present invention, the extract comprises or consists of an (preferably hydroalcoholic, in particular hydroethanolic) extract of flowers of Silybum marianum from which seeds have been removed, wherein said extract comprises less than 0.1% by weight of silymarin, or less than 0.01% by weight of silymarin, or less than 0.001% by weight of silymarin, or 0% by weight of silymarin, based on the total weight of the dry extract.

In a preferred embodiment, the flower extract of Silybum marianum is a hydroethanolic extract obtained by extraction flowers of Silybum marianum from which seeds have been removed with an ethanol/water mixture containing between 60 and 80% (v/v) ethanol (preferably at ambient temperature, e.g at 15 to 25° C.), wherein said extract comprises less than 0.1% by weight of silymarin, or less than 0.01% by weight of silymarin, or less than 0.001% by weight of silymarin, or 0% by weight of silymarin, based on the total weight of the dry extract.

As used herein, the term “dry extract” may be understood in the broadest sense as an extract according to the present invention as dry matter, i.e., without the solvent. The dry extract may optionally be physically present as dry matter. It will, however, be directly understood that, in calculations referring a percentage referred to dry matter, it does not have to be dry matter present in physical form, but the dry extract may also be present in dissolved form. Then, in the calculation, the mass of the solvent is mathematically subtracted from the total mass.

Dry matter (in physical form) may be obtained by a suitable extraction method, followed by a step of drying the extract obtained. The drying may be performed by any method suitable for this purpose known in the art. Drying means removing the one or more solvents used for extraction. Removing the one or more solvents may be performed by any means. For example, it may be achieved by subjecting the extract to a hot and dry atmosphere and/or placing the extract on a heated plate in order to evaporate the solvent. For example, drying may be achieved by evaporation in a vacuum and/or at elevated temperature (e.g., in a rotary (vacuum) evaporator) or by crystallization of the solid material of interest. Alternatively or additionally, drying may be achieved by out by atomisation or by lyophilisation.

According to the present invention, the flower extract of Silybum marianum may have any senotherapeutic activity. It may have reduced cellular senescence in a tissue and/or treat or prevent a disorder associated with cellular senescence in a tissue by any means.

In a preferred embodiment, the flower extract of Silybum marianum has senolytic activity and/or senomorphic activity.

In a preferred embodiment, the flower extract of Silybum marianum has senolytic activity including specifically inducing or promoting cell death of senescent cells in the tissue. In this context, cell death preferably is apoptotic cell death.

Preferably, the extract exerts senolytic activity. In other words, the flower extract of Silybum marianum is considerably as a senolytic agent or a senolytic component.

As used throughout the present invention, the term “senolytic” may be understood in the broadest sense as generally understood in the art. Senolytic activity refers to inducing or promoting cell death of senescent cells. It may be killing senescent cells. It may be eliminating senescent cells. Killing may be inducing or promoting apoptosis.

Preferably, senolytic activity refers to specifically (also: selectively) inducing or promoting cell death of senescent cells. This may also be considered as ability for eliminating selectively senescent cells, for example, by inducing apoptosis. It may also be the capability to selectively kill senescent cells found in a tissue with a reduced or even no harmful effect on the normal resident (i.e., non-senescent) cells of the said tissue. Such senolytic property may be beneficial, for example, for preventing or attenuating the increase of the number of age-related senescent cells and/or stress-related senescent cells within a tissue.

As used herein, the expression “specifically inducing or promoting cell death of senescent cells” may be understood in the broadest sense as having a stronger activity on senescent cells than on corresponding (i.e., comparable) non-senescent cells. Herein the terms “specifically” may also be selectively. Corresponding and, thus, comparable cells are typically of the same tissue type origin and the essentially only different is the degree of senescence. In a preferred embodiment, inducing or promoting cell death effects senescent cells more than 1.05-fold, or more than 1.1-fold, or more than 1.2-fold, or more than 1.3-fold, or more than 1.4-fold, or more than 1.5-fold, or more than 2-fold, in comparison to the corresponding (i.e., comparable) non-senescent cells.

In a preferred embodiment, the flower extract of Silybum marianum has senomorphic activity including suppressing senescence phenotypes in senescent cells in the tissue, restoring metabolic process in the senescent cells in the tissue, or a combination of two or all thereof.

Preferably, the extract exerts senomorphic activity. In other words, the flower extract of Silybum marianum is considerably as a senomorphic agent or a senomorphic component.

As used throughout the present invention, the term “senomorphic” may be understood in the broadest sense as generally understood in the art. Senomorphic activity may include or may be based on suppressing senescence phenotypes in senescent cells in the tissue. It may include the ability for inhibiting the senescence-associated secretory phenotype (SASP) factor secretion without killing the senescent cells and/or for reversing the proliferative arrest of the senescent cells and/or for restoring its ability to maintain normal metabolic process of the senescent cells, particularly senescent skin and hair follicle cells. Senomorphic activity may include modulating or even revering the phenotypes of senescent cells to those of young cells (or non-senescent cells) by interfering with trigger of senescent cells, targeting senescent cells directly, reducing or blocking SASP factor secretion, and/or promote the growth ability previously lost by those said senescent cells. Senomorphic activity may include suppressing or decreasing SASP factor secretion by senescent cells, and/or to restore the ability to maintain normal metabolic process of the said senescent cells in order to extend health span and potentially lifespan. Senomorphic activity may be beneficial, for example, for preventing and/or treating cellular senescence-associated disorders within a tissue, particularly in skin and hair. The composition of the present invention may be used for selectively killing the senescent cells and/or for inhibiting SASP-release from senescent cells, and/or for regenerating cell proliferation ability, and/or for counteracting senescence-induced cell deterioration, and/or for restoring metabolic process in senescent cells or a mixture thereof.

As used herein, examples of SASP factors may be selected from the group consisting of tumor necrosis factor alpha (TNF-α), inflammatory cytokines such as IL-1α, IL-1β, IL-6, IL-8; metalloproteinases such as MMP-1 and MMP-3 and γ-H2AX.

The flower extract of Silybum marianum may optionally also serve as a mediator of the immune-system allowing (partial) clearance of senescent cells.

In a preferred embodiment, the flower extract of Silybum marianum has both, senolytic and senomorphic activity.

In a preferred embodiment, the flower extract of Silybum marianum has one or more of the following activities on the senescent cells in the tissue selected from the group consisting of:
(a) inhibiting release of senescence-associated secretory phenotype (SASP) factors from senescent cells in the tissue, in particular wherein SASP factors are selected from the group consisting of gamma-H2AX, tumor necrosis factor alpha, inflammatory cytokines such as interleukins (ILs) selected from the group consisting of IL-1α, IL-1β, IL-6, and IL-8, metalloproteinases such as matrix metalloproteinases (MMPs) selected from the group consisting of MMP-1 and MMP-3;
(b) regenerating cell proliferation ability of the senescent cells;
(c) counteracting senescence-induced cell deterioration of the senescent cells;
(d) restoring metabolic process in the senescent cells, and
(e) inducing or promoting cell death of senescent cells.

According to a preferred embodiment of the present invention, a flower extract of Silybum marianum or a cosmetic composition containing said extract may be suitable and/or intended for use in selectively killing the senescent skin and hair follicle cells, and/or in inhibiting SASP-release from senescent skin and hair follicle cells, and/or in regenerating skin and hair follicle cell proliferation ability, and/or in counteracting senescence-induced skin and hair follicle deterioration, and/or in restoring metabolic process in senescent skin and hair follicle cells or a mixture thereof.

In a preferred embodiment, the composition comprises 0.0001 to 20% by weight, preferably 0.001 to 15% by weight, more preferably 0.005 to 10% by weight, in particular 0.01 to 5% by weight by weight, based on the total weight of the composition, of a flower extract of Silybum marianum.

The composition may comprise any further ingredients in addition to the flower extract of Silybum marianum. Preferably, such one or more further ingredients are suitable for uses in cosmetic and/or pharmaceutical products.

Thus, the composition according to the present invention may comprise:
(A) 0.0001 to 20% by weight, preferably 0.001 to 15% by weight, more preferably 0.005 to 10% by weight, in particular 0.01 to 5% by weight by weight, based on the total weight of the composition of an extract of flowers of Silybum marianum from which seeds have been removed; and
(B) at least one cosmetically and/or pharmaceutically acceptable carrier.

Herein, the flower extract of Silybum marianum may be considered as an (or the sole) active ingredient. The term “active ingredient” may be understood in the broadest sense as a component that may exhibit the desired and intended activity alone or may exhibit the activity together with one or more carriers that are themselves inactive. Preferably, the active ingredient of the present invention is a cosmetic active ingredient.

As used herein, the terms “pharmaceutically acceptable carrier”, “pharmaceutically acceptable excipient”, “cosmetically acceptable carrier”, “cosmetically acceptable excipient”, “carrier” and “excipient” may be understood interchangeably in the broadest sense as any substance that may support the cosmetic and/or pharmacological acceptance of the composition according to the present invention containing the flower extract of Silybum marianum.

Preferably, neither the flower extract of Silybum marianum nor a composition containing such extract according to the present invention is not toxic when applied to the tissue.

For each of the composition according to the present invention, techniques for formulation and administration are well known in the field of the invention.

The composition ready to use preferably is a liquid formulation, in particular a composition suitable for topic administration. The storage form of the composition may also be liquid, but may also be a dried form (e.g. a powder such as a powder comprising or consisting of the flower extract of Silybum marianum) or may be a paste or syrup or the like. Optionally, a dried form, paste or syrup may be dissolved or emulsified prior to being administered to the tissue of interest.

A cosmetically and/or pharmaceutically acceptable carrier may exemplarily be selected from the list consisting of an aqueous buffer, saline, water, alcohols, vegetable oils, mineral oils, polymers, gaz or combination of two or more thereof.

Optionally, a cosmetically and/or pharmaceutically acceptable carrier may contain one or more cosmetically and/or pharmaceutically acceptable additives. In a preferred embodiment, such cosmetically and/or pharmaceutically acceptable additives may be selected from the group consisting of fragrances/perfumes, dyes, pigments, emulsifiers, lubricants, chelating agents, acidity regulators, antimicrobial agents, preservatives, antioxidants, and combinations of two or more thereof. For instance, the cosmetically and/or pharmaceutically acceptable carrier may optionally contain one or more detergents, triethanolamine, one or more fragrances, one or more foaming agents (e.g., sodium lauryl sulphate (SLS), sodium doceyl sulphate (SDS)), one or more colouring agents (e.g., food colouring, pigments), one or more vitamins, one or more salts (e.g., sodium, potassium, calcium, zinc salts), one or more humectants (e.g., sorbitol, glycerol, butylene glycol, propylene glycol, mannitol, propylene glycol, polydextrose), one or more enzymes, one or more preserving agents (e.g., benzoic acid, methylparabene, one or more antioxidants, one or more herbal and plant extracts, one or more stabilizing agents, one or more chelating agents (e.g., ethylenediaminetetraacetic acid (EDTA), one or more polymers (e.g., carboxyvinyl polymer, carboxymethyl cellulose, cellulose, carboxyethyl cellulose), one or more stabilizers, one or more solubilizers, one or more emollients, and/or one or more uptake mediators (e.g., polyethylene imine (PEI), a cell-penetrating peptide (CPP), a protein transduction domain (PTD), an antimicrobial peptide, etc.).

Moreover, the composition of the present invention may contain, in addition to the flower extract of Silybum marianum, one or more other active ingredients, working either synergistically and/or complementarily with the flowers extract of Silybum marianum without affecting its activity or its indented use as mentioned above.

The composition according to the present invention may be cosmetic product or may be comprised in a cosmetic product. Deleterious effects in the skin may be reduced or avoided, according to the safety studies and legal requirements.

The composition may be for any use. Any administration routes are suitable that lead to the desired purpose as claimed. Administration may be local or systemic administration. Preferably, administration is local administration. Administration may be topic administration, transdermal administration, oral administration, administration by mean of injection (e.g., intravenous (i.v.), intraarterial (i.a.), intraperitoneal (i.p.), intramuscular (i.m.), and subcutaneous (s.c.) injection), or nasal administration. For example, the composition according to the present invention may be formulated, with acceptable carriers and excipients, in any suitable form for topical, oral, rectal, transmucosal, transnasal, intestinal, enteral and parenteral administrations.

In a preferred embodiment, the composition is a composition for topic use. In a preferred embodiment, the composition is a composition for topic use which is administered topically to a subject having the tissue. In other words, the composition according to the present invention may be administered topically. In a preferred embodiment, the composition is a composition for topic use which is administered topically to a part of the skin such as, e.g., to the face, scalp, or part of a body or into the hair. Skin may be all types of skin, including dry skin, normal skin and oily skin.

The flower extract of Silybum marianum may have a local and/or a systemic effect. In a preferred embodiment, when administered locally and topically, it (mainly or completely) has a local effect.

As used in the context of the present invention, the term “subject” may be understood in the broadest sense as any living being, which is preferably any animal, more preferably a mammal including human, in particular a human being.

The composition may be a composition for any use. Preferably, the composition is a cosmetic composition, a pharmaceutical composition, or a nutraceutical composition. In a preferred embodiment, the composition is selected from the group consisting of emulsions, gels, ointments, tonics, liquid soaps, bar soaps, bath oils, shower oils, massage oils, makeups, scalp treatments, aftershaves, shaving products, deodorants, shower gel, shampoos, and combinations of two or more thereof. In a preferred embodiment, the composition is a composition for topic use selected from the group consisting of a solution, a suspension, an emulsion, a cream, a paste, a gel, a lotion, a powder, a soap, a surfactant-containing water, an oil, and a spray.

In an alternative preferred embodiment, the composition is a nutraceutical composition which is administered orally to a subject having the tissue. The flower extract of Silybum marianum may be optionally included in a food product, such as a food supplement. Then, typically, the flower extract of Silybum marianum has a systemic effect. A nutraceutical composition according to the present invention may be formulated, with acceptable carriers or excipients, in any conventional form suitable for oral administration such as, e.g., tablets, capsules, granules, powder, solution, emulsion and suspension.

Cellular senescence in the context of the present application may be understood in the broadest sense. It may have any reason. In a preferred embodiment, cellular senescence occurs from the presence of age-related senescent cells, stress-related senescent cells, or both.

The tissue from which the senescent cells are originating from and/or in which the senescent cells are located may be any tissue. In a preferred embodiment, the tissue is selected from a group consisting of a tissue present in the skin, in particular a dermal tissue, an epidermal tissue, a subcutaneous tissue, a hair follicle tissue, and a combination of two or more thereof.

The intended reduction of cellular senescence may be strived for any cosmetic and/or therapeutic purpose. In a preferred embodiment of the present invention, reducing cellular senescence in the tissue is for improving the appearance or other properties of skin, hair, or both.

The senescent cells may be of any origin. In a preferred embodiment, the senescent cells originate from the skin, in particular a dermal tissue, an epidermal tissue, a subcutaneous tissue, a hair follicle tissue, and a combination of two or more thereof. In a preferred embodiment of the present invention, senescent cells are from tissues selected from skin, hair follicle and body. More generally, tissues may be constituted by cells from mesenchymatous origin. Herein, senescence may be induced by both intrinsic and extrinsic factors. Preferably, tissues may be selected from skin and/or hair follicles. In a preferred embodiment, senescent skin cells are selected from fibroblasts, melanocytes, keratinocytes, endothelial, and adipocytes. In a preferred embodiment, senescent cells are senescent skin cells selected from fibroblasts, melanocytes, keratinocytes, endothelial, and adipocytes.

In a preferred embodiment of the present invention, reducing cellular senescence in the tissue is for improving skin elasticity, skin firmness, skin thickness, skin tone, skin radiance, skin barrier, skin moisturizing, skin rejuvenation, or two or more thereof, or for counteracting or ameliorating skin sagging, skin hyperpigmentation, skin hypopigmentation, loss of skin elasticity and firmness, erythema, poor skin barrier function, DNA-damages, hair-loss, hair-thinning, hair fragility, grey-hair, or two or more thereof.

According, composition comprising flower extract of Silybum marianum may be used to treat or prevent or to improve skin elasticity, skin firmness, skin thickness, skin sagging, skin tone, skin radiance, skin barrier, skin moisturizing, skin rejuvenation, skin hyperpigmentation, skin hypopigmentation, loss of skin elasticity and firmness, erythema, poor skin barrier function, DNA-damages, hair-loss, hair-thinning, hair fragility, grey-hair, or a mixture thereof.

Some field in which the application may be used also refer to therapeutic/pharmaceutical uses.

Thus, a further aspect of the present invention also refers to a composition comprising or consisting of an extract of flowers of Silybum marianum from which seeds have been removed, as a senotherapeutic agent for use in a method for treating or preventing a disorder associated with cellular senescence in a tissue present in the skin.

It will be understood that the definitions and preferred embodiments made in the context of the cosmetic use of the present invention mutatis mutandis apply to the composition for use of the present invention.

In other words, the present application also refers to a method for treating or preventing a disorder associated with cellular senescence in a tissue present in the skin, wherein the tissue is administered with a sufficient amount of a composition comprising or consisting of flower extract of Silybum marianum (as a senotherapeutic agent) to said tissue.

As used herein an “agent” may be understood in the broadest sense as any component of the composition. It may be a complex component containing different chemical entities, i.e., compounds, or may be a component consisting of a single component. An extract of flowers of Silybum marianum according to the invention is preferably a complex component comprising a number of chemical entities. A senotherapeutic agent may also be designated as “senotherapeutic component”.

The present invention also refers to a cosmetic composition comprising an extract of flowers devoid of seeds of Silybum marianum for use as a senotherapeutic active ingredient in preventing and/or in treating age-related or stress-related senescent cells in the skin and hair follicle; and/or in treating cellular senescence-associated disorders in the skin and hair follicles.

The present invention also refers to a pharmaceutical composition comprising an extract of flowers of Silybum marianum from which seeds have been removed, for use as a senotherapeutic active ingredient in treating cellular senescence-associated disorders within a tissue present in the skin; and/or for attenuating age-related senescent cells within the said a tissue.

The present invention also refers to a nutraceutical composition comprising at least an extract of flowers of Silybum marianum from which seeds have been removed, for use as a senotherapeutic active ingredient in preventing and/or in treating age-related or stress-related senescent cells within a skin and hair; and/or in treating cellular senescence-associated disorders within a skin and hair.

The present application also refers to a method for treating or preventing a disorder associated with cellular senescence in a tissue present in the skin in a subject, wherein the said tissue is administered with a sufficient amount of a composition comprising or consisting of an extract of flowers of Silybum marianum from which seeds have been removed (as a senotherapeutic agent) to said tissue of said subject. The present application also refers to a method for treating or preventing a disorder associated with cellular senescence in a subject, wherein the tissue present in the skin is administered with a sufficient amount of a composition comprising or consisting of an extract of flowers of Silybum marianum from which seeds have been removed (as a senotherapeutic agent) to said subject.

The present invention also refers to an extract of flowers of Silybum marianum from which seeds have been removed (as an active ingredient) for use as a senotherapeutic active ingredient in preventing and/or in treating age-related or stress-related senescent cells within a tissue present in the skin; and/or in treating cellular senescence-associated disorders within the said a tissue.

In view of the aforementioned, the present invention also refers to an extract of flowers of Silybum marianum from which seeds have been removed for use as a senotherapeutic active ingredient in preventing and/or in treating age-related or stress-related senescent cells within a tissue present in the skin; and/or in treating cellular senescence-associated disorders within the said a tissue.

The disorder to be treated may be any disorder associated with cellular senescence. As used herein, the terms “disorder” may be understood in the broadest sense as any condition to be treated (e.g., pathological condition). It may also be an inherent or an acquired disease.

A subject having the tissue of interest may be suffering from or may be of risk of developing the disorder. The term “suffering from” as used herein may be understood in the broadest sense in a way that the subject has developed a (pathological) condition associated. The subject suffering from a disorder not necessarily, but optionally, bears medicinal symptoms. The term “being at risk of” or “being at risk of developing” means that the subject has a certain risk of having a disorder in the sense of the present invention. In the therapeutic context, a subject may also be designated as “patient”.

In a preferred embodiment of the present invention, the disorder is selected from the group consisting of (pathological) skin dryness, (pathological) hyperpigmentation, (pathological) hypopigmentation, (pathological) loss of skin elasticity and firmness, erythema, (pathological) hair-loss, hair-thinning, hair fragility, grey-hair, and tissue-fibrotic disease.

The disorder may also be a cellular senescence-associated disorder within a tissue. As used herein, the term “cellular senescence-associated disorders within a tissue” may include any physiological disorders in a tissue including, for instance, skin disorders defined above, and tissue fibrotic disease.

The disorder may also be a cellular senescence-associated disorder in skin and hair within a tissue. As used herein, the term “cellular senescence-associated disorders in skin and hair” may be understood in the broadest sense as physiological disorders in these tissues due to, for example, the accumulation or increase amount of the senescent skin and hair follicle cells compared to non-senescent (normal) cells within the said tissues.

Disorders may be induced by both intrinsic and extrinsic factors such as time, genetic factors, hormones, lights, UV-exposure, pollutions; more generally external and environmental factors. Examples of disorders may be wrinkles, fine-lines, sagging, dryness, dullness, hyperpigmentation, hypopigmentation, loss of skin elasticity and firmness, erythema, poor skin barrier function, DNA-damages, hair-loss, hair-thinning, hair fragility and grey-hair.

It was surprisingly found that an extract containing less than 0.01% by weight of silymarin is particularly beneficial for the above purposes.

It will be understood that the definitions and preferred embodiments made in the context of the cosmetic use and the composition for use of the present invention mutatis mutandis apply to the composition of the present invention.

Throughout this specification and the claims, unless the context requires otherwise, the open-worded terms such as “comprise”, “contain”, “include”, etc., and variations such as “comprises”, “contains”, “includes”, “comprising”, “containing”, “including”, etc., maybe understood to imply the inclusion of a stated component, member, integer or step or group of components, members, integers or steps, but not the exclusion of any other components.

The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually as well as in combination with each other recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

All elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Unless defined otherwise, all technical and scientific terms used herein have the same meaning as commonly understood by a person skilled in the art. Although the methods and materials described herein are preferred, other methods and materials similar or equivalent to those described herein can be used in the practice or testing of the present invention as well.

All documents cited or referenced herein are hereby incorporated by reference, and may be employed in the practice of the invention. More specifically, all referenced documents are incorporated by reference to the same extent as if each individual document was specifically and individually indicated to be incorporated by reference.

The following Examples and Figures are intended to illustrate further embodiments of the present invention without limiting its scope.

### EXAMPLES

1. Preparation of Silybum marianum Flower Extract

The flowers of Silybum marianum where seeds have been removed, were dried and pulverized. Then, 100 g of the pulverized Silybum marianum powder was extracted by hydroalcoholic extraction overnight with 70% (v/v) ethanol (and 30% (v/v) water) at room temperature. The supernatant was collected by filtration. Ethanol and water were removed from collected supernatant by means of rotary vacuum evaporation (Heidolph, Schwabach, Germany). The extract obtained was lyophilized to yield Silybum marianum flower extract (SME).

### 2. In Vitro Comparative Study

In this study, the senotherapeutic effect of the flower extract of Silybum marianum (SME) was demonstrated. The senotherapeutic effect of the extract of Silybum marianum may be due to its senolytic activity and/or its senomorphic activity on senescent skin and hair follicle cells.

### Materials:

Reagents were obtained as follows: agent “ABT-737” which is commonly known as senolytic agent was purchased from abcam (MA, USA.); 3-(4, 5-dimethyl-2-thiazolyl)-2, 5-diphenyltetrazolium bromide (MTT), dimethyl sulfoxide (DMSO) were purchased from Duchefa (Haarlem, The Netherlands); Hoechst 33342, dead cell apoptosis kit was purchased from Invitrogen (CA, USA); antibodies and senescence β-Galactosidase staining kit were obtained from CST (MA, USA).

Induction of senescent skin senescent cells phenotype Non-senescent human dermal fibroblasts cells (HDFs) were obtained from ATCC PCS-201-010™ (VA, USA) and cultured in Dulbecco's Modified Eagle's Medium (DMEM; Hyclone, UT, USA) with 10% (v/v) foetal bovine serum and penicillin/streptomycin (100 IU/50 μg/mL) in a humidified atmosphere containing 5% CO₂ in air at 37° C. Non senescent HDFs from passages 8 to 10 were used for the experiments, with a confluence degree between 80 to 90%. Senescent HDFs was induced by cumulative population doubling (CPD) corresponding to 40 passages. Cells acquired the senescence phenotype after 40 passages.

Induction of hair follicle dermal papilla senescent cells phenotype (HFDPC) The hair follicle dermal papilla cells senescent were induced by cumulative population doubling (CPD) corresponding to 18 passages. HFDPC acquired the senescence phenotype after 18 passages.

### Cell Viability Assays

The non-senescent and senescent HDFs were seeded on a 24-well plate, respectively. After incubation with treatment of test materials (meaning by SME, ABT-737, etc.) for 3 days, HDFs viability was determined using MTT assay.

### SA-β-Galactosidase Staining Assay (Senescent Cells Markers)

HDFs and HFDPC were seeded at a constant cell density in a 6-well plate. After incubation with treatment of test materials for 3 days, the cells were stained using a senescence β-galactosidase staining kit (CST, MA, USA). SA-β-gal is used to assess the abnormal enzymatic activity observed in senescent cells. The culture was incubated for overnight after adding a staining solution; SA-β-gal in senescent cells stains blue. The proportion of senescent cells was computed as the number of senescent cells stained blue divided by the total number of cells. The average number of stained and total cells was determined.

Immunofluorescence staining of apoptotic markers and SASP markers HDFs were seeded on a 24-well plate. After incubation with treatment of test materials for 3 days, HDFs were fixed with 4% (w/v) formaldehyde for 15 min at room temperature and blocked with 5% bovine serum albumin, 0.3% (w/v) Tween 20 in PBS for 1 hour. The antibodies used caspase-3, PARP, IL-6 and IL-8 (1:50 dilution; CST, USA) was added to each well, followed by overnight at 4° C. Cells were washed with PBS and incubated with Hoechst 33342 (1:10000; Invitrogen, USA) for 10 min at room temperature. Fluorescence was subsequently analysed by fluorescence microscopy (EVOS; Life Technologies GmbH, Germany).

### Enzyme-Linked Immunosorbent Assay (ELISA) of SASP Markers

The level of MMP-1 in the cell supernatants was measured by Human matrix metalloproteinase 1 (MMP-1) Quantikine ELISA Kit (R&D system Inc., USA) according to the manufacturer's protocol. HDFs were treated with various concentrations of SME 100 and 200 ppm for 3 days and cell supernatants were collected after SME treatment.

### Statistical Analysis:

All data were expressed as mean±standard deviations. The statistical significance of the data was determined by two-way analysis of variance. *p<0.05, ** p<0.01, were indicated statistically significant.

### Results:

Results were obtained by performing 3 independent assays.

## p<0.01 compared to non-senescent cells control

* p<0.05; ** p<0.01; *** p<0.001, compared to senescent cells control

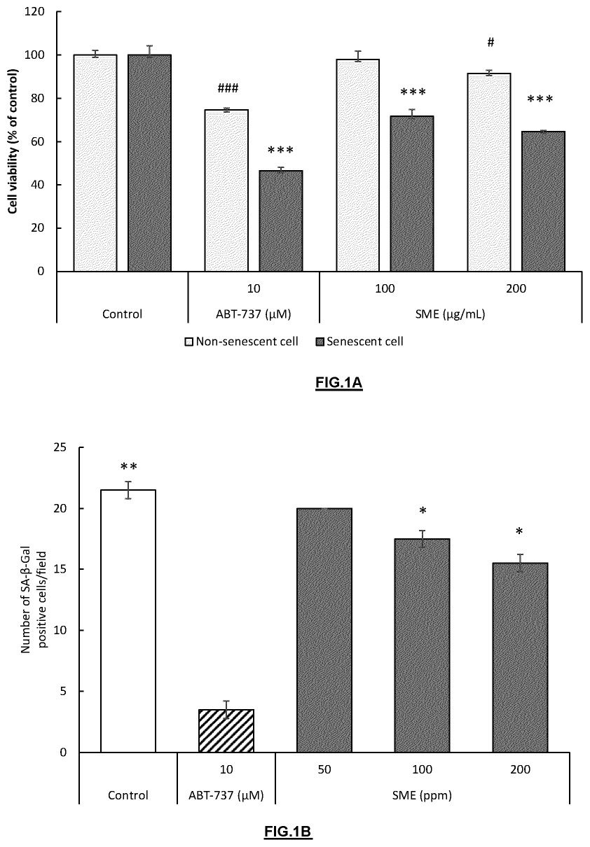

In order to investigate whether SME has a senolytic activity on senescent HDFs (FIG. 1), the cell viability in senescent and non-senescent HDFs was observed.

The senescent and non-senescent HDFs were treated with SME for 3 days. In order to validate the experimental assay, the effect of a known senolytic chemical compound (ABT-737) compared to the senolytic effect of SME (at 100 and ppm) on senescent HDFs (at least 40 passages in culture) was determined (FIG. 1A). Indeed, 3 days of treatment demonstrated a significant specific senolytic effect of SME on senescent HDFs. Cell viability was reduced by 35% in SME-treated senescent HDFs and no change was observed in non-senescent HDFs confirming the specific senolytic activity of SME on senescent HDFs.

Furthermore, in FIG. 1B, the SA-β-Gal activity was investigated in this senescence model of HDF treated by ABT-737 as positive control and SME at 50, 100 and ppm. After 3 days of treatment by SME, SA-β-Gal staining was significantly decreased in dose-dependent manner, confirming the ability of SME to affect the number of senescent HDFs. Moreover, the expression of the senescence marker p21 in non-senescent and senescent HDFs treated or not by SME at 100 ppm and ppm for 3 days was quantified (FIG. 1C). The strong expression of p21 in non-treated senescent HDF confirms the relevance of studying this cell cycle inhibitor as senescence marker. 3 days of treatment by SME demonstrated a dose-dependent inhibition of p21 expression in senescent HDFs, and demonstrating the senomorphic activity of SME.

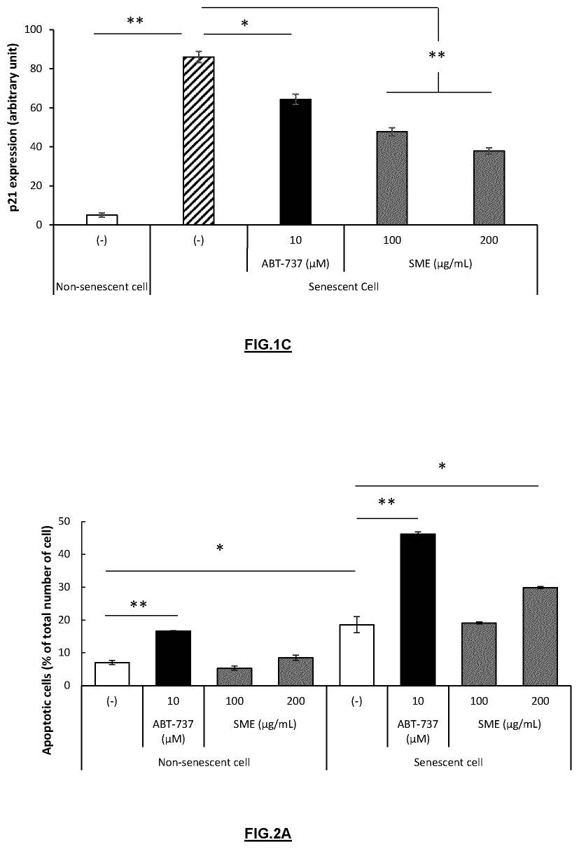

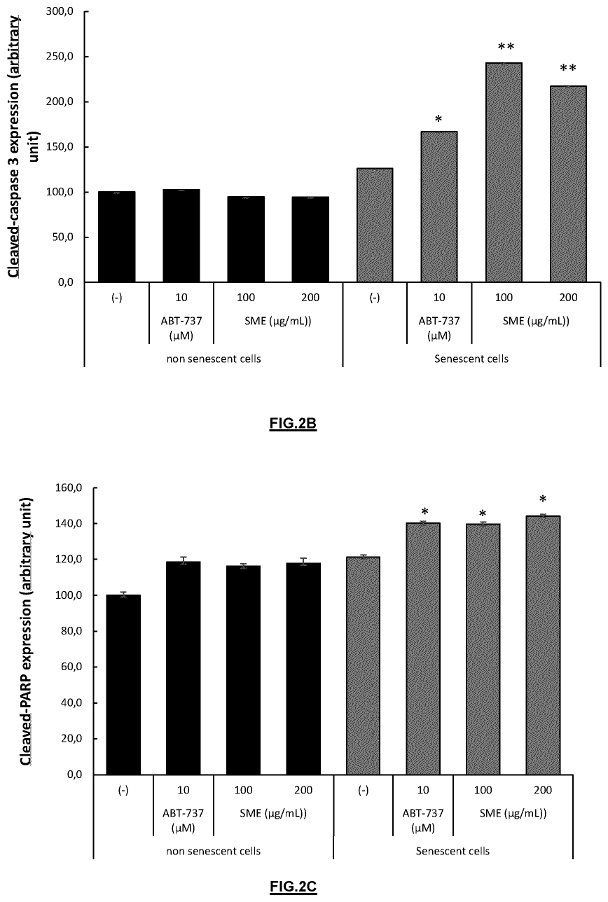

In order to determine the mechanism of action of the senolytic effect, apoptotic cells were investigated by means of a flow cytometer after after annexin V and propidium iodide (P1) staining. Quantification of the percentage of apoptotic HDF (annexin V-FITC positive) 3 days after treatment was performed (FIG. 2A). A known apoptotic compound was used to validate the assay. In non-senescent HDF, 3 days of treatment by SME did not modulate the number of apoptotic HDF, demonstrating a non-toxicity of SME for non-senescent HDFs. Moreover, SME at ppm significantly increased the number of apoptotic HDF in replicative model senescent HDF, confirming the senolytic activity of SME showed in FIG. 1A and FIG. 1B.

In order to confirm that the induced-apoptosis is specific to senescent HDF, the caspase-3 expression within non-senescent and senescent HDF was determined. Indeed, caspase-3 plays a central role in the execution-phase of cell apoptosis and its proteolytic cleavage leads to its activation. The cleavage inhibition of poly-ADP-ribose polymerase (PARP) (mediated by caspase) facilitates cellular disassembly and serves as a marker of cells undergoing apoptosis. FIGS. 2B and 2C show the occurrence of cleaved caspase-3 and cleaved PARP, respectively, after SME treatment for 3 days at SME concentrations of 100 ppm and 200 ppm. Quantification of fluorescence intensity analysis of cleaved caspase-3 (FIG. 2B) and cleaved PARP (FIG. 2C) were obtained. The treatment of senescent HDF by SME for 3 days significantly increased the cleavage of caspase-3 and PARP, confirming the specific induction of apoptosis in senescent HDFs by SME, demonstrating the senolytic activity of SME.

The aforementioned data demonstrate that SME enables selectively killing senescent cells (exemplified as human dermal fibroblasts cells (HDFs)) by inducing them into apoptosis.

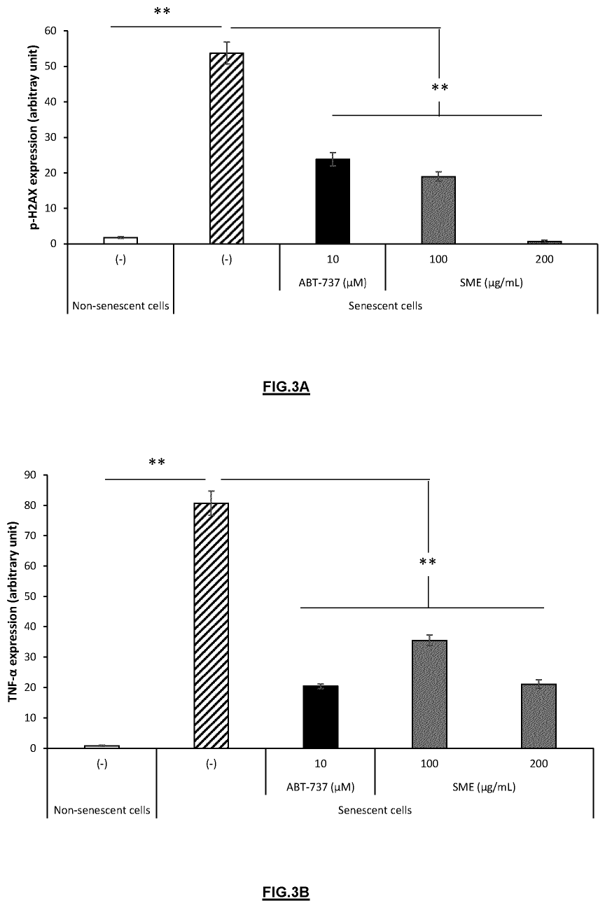

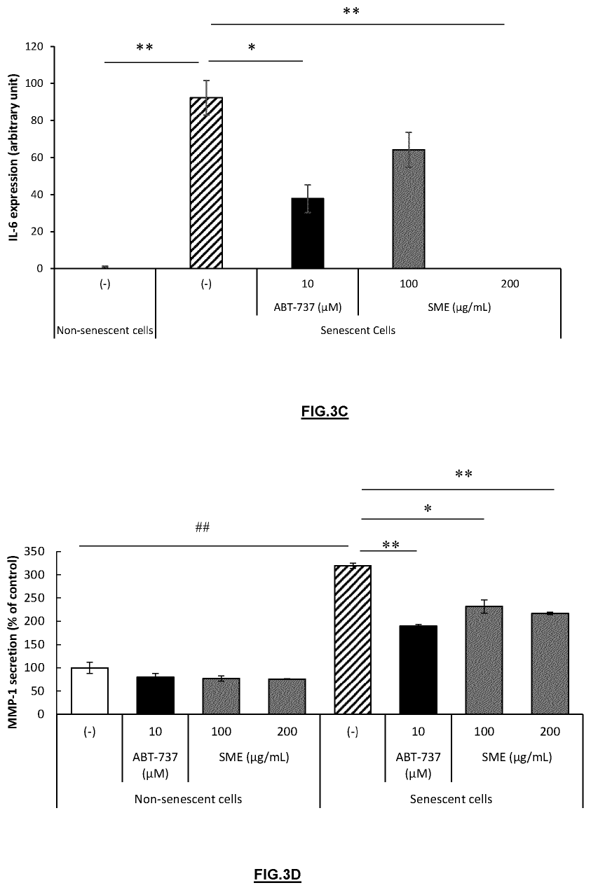

In order to investigate the senomorphic activity of SME, immunofluorescence intensity measurements of specific SASP markers and SASP regulators were performed. The senomorphic activity of SME in senescence replicative model of HDF by inhibition of SASP factors release was reported (FIG. 3). FIGS. 3A, 3B and 3C demonstrate the quantification of immunofluorescence intensity measurements, corresponding respectively to γ-H2AX marker, TNF-α and IL-6 cellular expression. The secretion of MMP-1 is represented in FIG. 3D and measured by ELISA assay. The effect of SME on senescent HDF on the expression levels of DNA-damage response marker γ-H2AX known to regulate SASP factors release at SME concentrations of 100 ppm and 200 ppm was investigated (FIG. 3A). Senescent HDFs bear a strong increase of γ-H2AX expression compared to non-senescent HDF. This potent expression was significantly counteracted after 3 days of treatment by SME which induced a dose-dependent decrease of γ-H2AX expression in senescent HDFs.

Additionally, FIGS. 3B and 3C show the effect of SME at SME concentrations of ppm and 200 ppm on senescent HDFs on the expression levels of SASP factors, exemplified as TNF-α and IL-6, respectively. Senescent HDFs bear a strong increase of TNF-α and IL-6 expression compared to non-senescent HDFs. These increased expressions were significantly counteracted after 3 days of treatment by SME which induced a dose-dependent decrease of these SASP factors expression in senescent HDFs.

Furthermore, FIG. 3D shows the MMP-1 secretion level in supernatant of culture of non-senescent or senescent HDF treated or not by SME at 100 ppm and 200 ppm for 3 days. ABT-737 was used as positive control. MMP-1 is a well-known SASP factor involved in senescence-associated dermal matrix degradation. MMP-1 secretion was significantly increasing in senescent HDFs compared to non-senescent HDFs. This potent secretion was significantly counteracted after 3 days of treatment by SME which induced a decrease of the MMP-1 secretion by senescent HDFs.

The aforementioned results demonstrate the senomorphic activity exerted by SME on senescent cells (exemplified as HDFs) by inhibiting the SASP secretion.

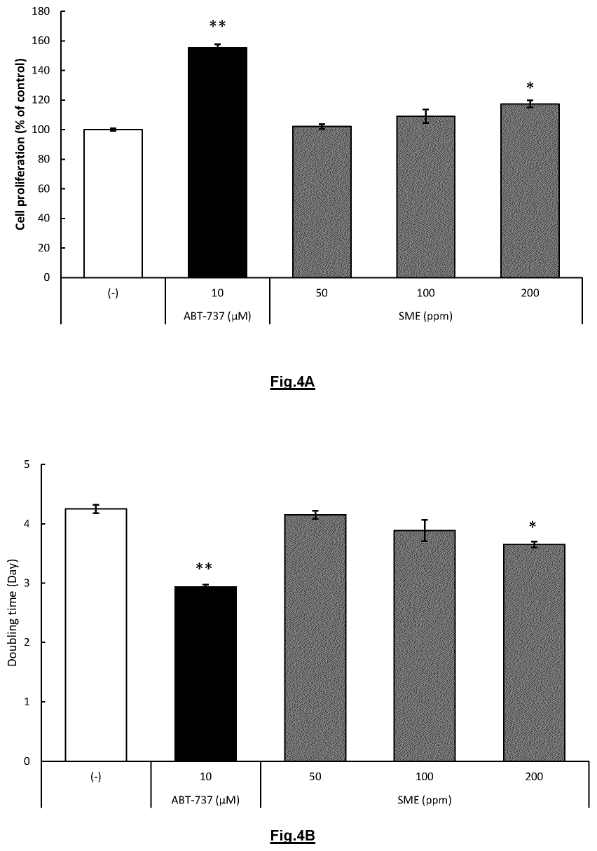

In order to investigate further senomorphic activity of SME, senescence replicative model of HDF was used for determining whether SME enabled to restore the proliferation ability of the senescent HDFs (FIG. 4). For this purpose, the assessment of SME capacity to stimulate the cell proliferation following the removal of senescent HDFs was allowed by the differential staining of senescent and non-senescent HDFs. Senescent and non-senescent HDFs were mixed at the ratio 1:9 respectively, and treated with the SME for 3 days, then cells were harvested and re-seeded. After 3 days of incubation, cell growth rate and population doubling time measured by cell counting. Results of FIG. 4A and FIG. 4B were measured by cell counting.

FIG. 4A shows the cell growth rate of mixed senescent and non-senescent HDFs after SME treatment at SME concentrations of 50, 100 and 200 ppm, respectively. Indeed, SME increases the proliferation rate significantly at a SME concentration of 200 ppm, demonstrating its ability to improve the regeneration of skin after the removal of senescent HDFs. Consecutively, FIG. 4B demonstrates the effect of SME on the doubling time of mixed senescent and non-senescent HDF after 3 days of treatment by SME. Indeed, SME decreases the population doubling time significantly at a SME concentration of 200 ppm, confirming its ability to improve the regeneration of skin after the removal of senescent HDFs.

The aforementioned results demonstrate the senomorphic activity exerted by SME on senescent HDFs by improving both, the proliferation rate and the doubling time.

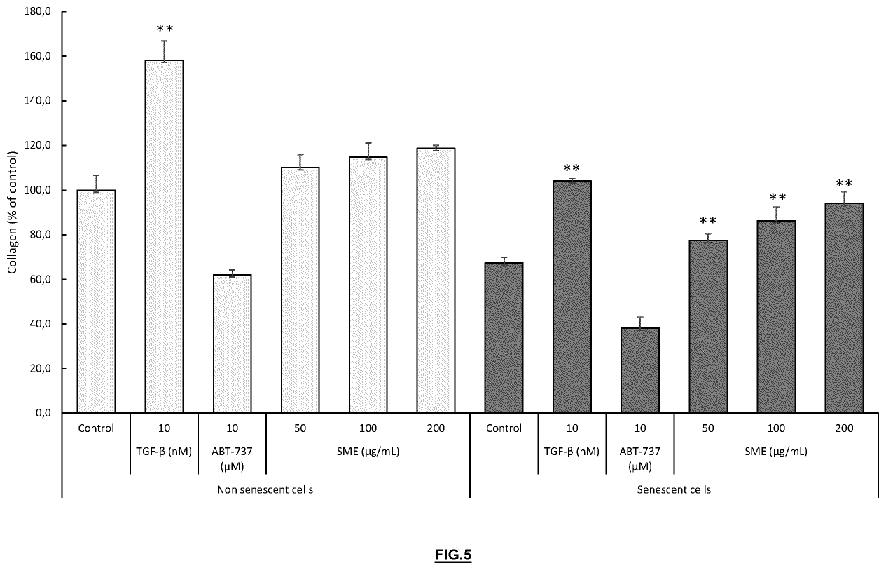

In order to demonstrate the senomorphic activity of SME, a senescence replicative model of HDFs was used to determine whether SME enables restoring the ability of senescent HDF in synthetizing collagen type I. For this purpose, an ELISA kit was used for assessing this effect by means of measuring the amount of collagen type I in the cell supernatants. FIG. 5 shows that SME, at concentrations of 50, 100 and 200 ppm, increased in dose dependent manner the collagen type I synthesis in senescent HDFs compared to non-senescent HDFs. This result confirms that, in addition to the ability of SME to selectively kill senescent HDF (indicating a senolytic activity), it shows that SME also enables restoring the ability of senescent HDF to synthetize collagen type I (indicating a senomorphic activity). This effect seems to be specific to senescent cells as the stimulation of collagen type I was not observed on senescent cells. Therefore, SME may be used for skin rejuvenation, skin repair of aged skin and as anti-wrinkle agent by targeting senescent cells.

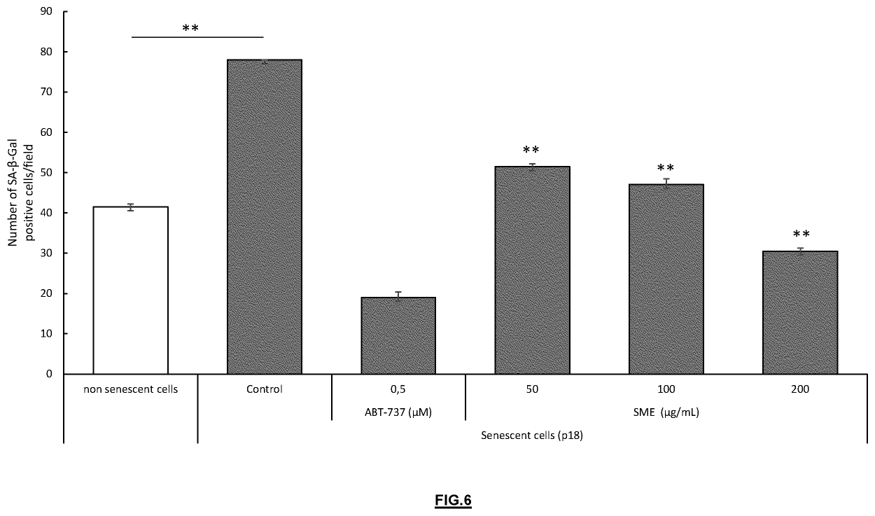

Furthermore, SA-β-Gal activity was investigated in this senescence model of hair follicle dermal papilla cells treated by ABT-737 as positive control and SME at concentrations of 50, 100 and 200 ppm (FIG. 6). After 3 days of treatment by SME at 200 ppm, SA-β-Gal staining was significantly decreased in dose-dependent manner, confirming the ability of SME to affect the number of senescent hair follicle dermal papilla cells, thus confirming the senolytic activity. This demonstrates that the positive effects can be used in various cell types.

In view of the above, it was experimentally found that an extract of Silybum marianum (SME) of the present invention can be very well used as a senotherapeutic agent. In particular, it was found that such extract bears senolytic and senomorphic effects. Cell death in senescent cells was selectively induced and promoted, respectively, in particular by means of inducing and promoting apoptosis of the senescent cells. Furthermore, senescence phenotypes could be suppressed in surviving senescent cells by means of treatment with the extract of Silybum marianum (SME).

Accordingly, it was shown that such extract may be used for reducing cellular senescence in the tissue is for improving the appearance or other properties of skin, hair, or both, in particular is for improving skin elasticity, skin firmness, skin thickness, skin tone, skin radiance, skin barrier, skin moisturizing, skin rejuvenation, or two or more thereof, as well as for counteracting or ameliorating skin sagging, skin hyperpigmentation, skin hypopigmentation, loss of skin elasticity and firmness, erythema, poor skin barrier function, DNA-damages, hair-loss, hair-thinning, hair fragility, grey-hair, or two or more thereof, as well as for treating or preventing dryness, hyperpigmentation, hypopigmentation, loss of skin elasticity and firmness, erythema, hair-loss, obesity, diabetes, neurodegenerative diseases, osteoarthritis, atherosclerosis, age-related eye diseases, and tissue-fibrotic disease.

### 3. Cosmetic Formulation Example

A cream comprising Silybum marianum flower extract (SME) suitable for a topical application is prepared according to a conventional method:

TABLE 1
Composition of a cream comprising Silybum marianum
flower extract (SME), sum of components: 100%
by weight

| | Content |
|---|---|
| Component | (% by weight) |
| Silybum marianum flower extract (SME) | 0.01 |
| Glycerol | 3.0 |
| Butylene glycol | 2.0 |
| Propylene glycol | 2.0 |
| Carboxyvinyl polymer | 0.1 |
| Ethanol | 10.0 |
| Triethanolamine | 0.1 |
| Preservatives, pigment, fragrance, | 82.79 |
| purified water | |

1.-13. (canceled)

14. A method for treating cellular senescence-associated disorders in skin and/or hair follicles by applying, a composition comprising an extract of flowers of Silybum marianum from which seeds have been removed, comprising less than 0.1% by weight of silymarin, based on the total weight of the dry extract, onto the skin and/or the hair follicles having senescence-associated disorders.

15. The method for treating cellular senescence-associated disorders in the skin and/or hair follicles according to claim 14, wherein the extract of Silybum marianum is a hydroalcoholic or an alcoholic extract

16. The method for treating cellular senescence-associated disorders in the skin and/or hair follicles according to claim 14, wherein the extract of Silybum marianum is a hydroalcoholic extract.

17. The method for treating cellular senescence-associated disorders in the skin and/or hair follicles according to claim 14, wherein the extract of Silybum marianum has senolytic activity including specifically inducing or promoting cell death of senescent cells in the skin and/or the hair follicles with senescence-associated disorders.

18. The method for treating cellular senescence-associated disorders in the skin and/or hair follicles according to claim 19, wherein the extract of Silybum marianum has senolytic activity including specifically inducing or promoting apoptotic cell death of senescent cells in the skin and/or the hair follicles with senescence-associated disorders.

19. The method for treating cellular senescence-associated disorders in the skin and/or hair follicles according to claim 14, wherein the extract of Silybum marianum has senomorphic activity including suppressing senescence phenotypes in senescent cells in the skin and/or hair follicles, restoring proliferation ability of senescent in the skin and/or hair follicles, restoring metabolic process in the senescent cells in the skin and/or hair follicles, or a combination of two or all thereof.

20. The method for treating cellular senescence-associated disorders in the skin and/or hair follicles according to claim 14, wherein the extract of Silybum marianum has one or more of the following effects on the senescent cells in the skin and/or hair follicles selected from the group consisting of:
(a) inhibiting release of senescence-associated secretory phenotype (SASP) factors from senescent cells in the skin and/or hair follicles;
(b) regenerating cell proliferation ability of the senescent cells;
(c) counteracting senescence-induced cell deterioration of the senescent cells;
(d) restoring metabolic process in the senescent cells, and
(e) inducing or promoting cell death of senescent cells.

21. The method for treating cellular senescence-associated disorders in the skin and/or hair follicles according to claim 20, wherein the SASP factors are selected from the group consisting of gamma-H2AX, tumor necrosis factor alpha, inflammatory cytokines such as interleukins (ILs) selected from the group consisting of IL-1α, IL-1β, IL-6, and IL-8, and metalloproteinases such as matrix metalloproteinases (MMPs) selected from the group consisting of MMP-1 and MMP-3.

22. The method for treating cellular senescence-associated disorders in the skin and/or hair follicles according to claim 14, wherein the composition comprises 0.0001 to 20% by weight of the extract of Silybum marianum, based on the total weight of the composition.

23. The method for treating cellular senescence-associated disorders in the skin and/or hair follicles according to claim 14, wherein the composition is a composition for topic use which is administered topically to a subject having the cellular senescence-associated disorders in the skin and/or hair follicles or wherein the composition is a nutraceutical composition which is administered orally to a subject having the cellular senescence-associated disorders in the skin and/or hair follicles.

24. The method for treating cellular senescence-associated disorders in the skin and/or hair follicles according to claim 23, wherein the composition is a composition for topic use selected from the group consisting of a solution, a suspension, an emulsion, a cream, a paste, a gel, a lotion, a powder, a soap, a surfactant-containing water, an oil, and a spray.

25. The method for treating cellular senescence-associated disorders in the skin and/or hair follicles according to claim 14, wherein the cellular senescence occurs from the presence of age-related senescent cells, stress-related senescent cells, or both.

26. The method for treating cellular senescence-associated disorders in the skin and/or hair follicles according to claim 14, wherein the skin and/or hair follicles present in the skin is selected from an epidermal tissue, a subcutaneous tissue, a hair follicle tissue, and a combination of two or more thereof.

27. The method for treating cellular senescence-associated disorders in the skin and/or hair follicles according to claim 14, wherein the method reduces cellular senescence in the cellular senescence-associated disorders in the skin and/or hair follicles by improving the appearance or other properties of skin, hair, or both.

28. The method for treating cellular senescence-associated disorders in the skin and/or hair follicles according to claim 27, wherein the method is for improving skin elasticity, skin firmness, skin thickness, skin tone, skin radiance, skin barrier, skin moisturizing, skin rejuvenation, or two or more thereof, or for counteracting or ameliorating skin sagging, skin hyperpigmentation, skin hypopigmentation, loss of skin elasticity and firmness, erythema, poor skin barrier function, DNA-damages, hair-loss, hair-thinning, hair fragility, grey-hair, or two or more thereof.

29. A composition comprising an extract of flowers of Silybum marianum from which seeds have been removed, comprising less than 0.1% by weight of silymarin, based on the total weight of the dry extract.
